(19) United States
(12) Patent Application Publication
(10) Pub. No.: US 2007/0250149 A1
(43) Pub. Date: Oct. 25, 2007

(54) Stiffening Support Catheters and Methods for Using the Same

(75) Inventors: Randolf Von Oepen, Los Altos Hills, CA (US); Marc Gianotti, Wiesendangen (CH)

Correspondence Address: WORKMAN NYDEGGER, 1000 EAGLE GATE TOWER,, 60 EAST SOUTH TEMPLE, SALT LAKE CITY, UT, 84111, US

(73) Assignee: ABBOTT LABORATORIES, Abbott Park, IL (US)

(21) Appl. No.: 11/738,386

(22) Filed: Apr. 20, 2007

Related U.S. Application Data
(60) Provisional application No. 60/793,781, filed on Apr. 21, 2006.

(51) Int. Cl. A61F 2/84

(52) U.S. Cl. 623/1.11

(57) ABSTRACT

A delivery system catheter that can track to a deployment location and has sufficient stiffness to support the catheter within the anatomy, thereby aiding with deployment of a stent with minimal movement of the stent. This results in more accurate placement of the stent at the deployment location. The delivery system catheter includes a catheter body having a proximal end, a distal end, and an interior wall surface defining a first lumen extending from the proximal end toward the distal end. A second lumen is disposed between the interior wall surface and an outer surface of the catheter body. A plurality of stiffening members is disposed around the second lumen, the plurality of stiffening members being engageable to selectively stiffen the catheter body.

### CROSS-REFERENCE TO RELATED APPLICATIONS

This application claims the benefit of the U.S. Provisional Patent Application No. 60/793,781, filed Apr. 21, 2006, and entitled “Medical Devices,” which is incorporated herein by reference in its entirety. Further, this application is related to commonly-assigned U.S. patent application Ser. No. ______, having Attorney Docket No. 17066.38.1, entitled “Stiffening Support Catheter And Methods for Using The Same,” filed on Apr. 20, 2007, which is incorporated herein by reference in its entirety.

### BACKGROUND OF THE INVENTION

I. Field of the Invention

The present invention generally relates to the field of medical devices. More specifically, the present invention relates to delivery system catheters or catheters that can be manipulated.

II. Related Technology

The use of intravascular devices to treat cardiovascular diseases is well known in the field of medicine. The need for a greater variety of devices to address different types of circumstances has grown tremendously as the techniques for using intravascular devices has progressed. One type of intravascular device is a catheter. Typically, an intravascular catheter is delivered into the body by routing it through the proper vessels in the body's vascular network in order to arrive at a site in need of a diagnostic or therapeutic technique.

The use of stents to treat cardiovascular diseases also is well known in the field of medicine. In general, a stent is use to counteract significant decreases in vessel or duct diameter, often to alleviate blood blow to organs beyond an obstruction in order to maintain an adequate delivery of oxygen. Although most commonly used in coronary arteries, stents also are widely used in other tubular structures such as central and peripheral arteries and veins, bile ducts, the esophagus, colon, trachea, or large bronchi, ureters, and urethra. Specifically, a stent is either an expandable form or perforated tube that is inserted into one of the above tubular conduits to prevent or counteract the flow constriction, which usually is caused by disease. Treatment of vascular disease, for example, often involves the deployment of stents within tortuous vessels by means of stent delivery systems or delivery system catheters. This can result in difficulties and inconsistencies with the deployment of the stent (or stents) because the vessel is acting to deform the stent and stent delivery system as it is being deployed, and vice versa. These loads can result in misplacement of the stent or movement of the stent from the treatment site during delivery. For example, in the case of treating carotid arteries, the guidewire travels from the aortic arch into the opening of the carotid artery (the carotid artery ostium). This passage may be complicated by the severity of the arch and the placement of the ostium. In the worst cases where difficult angles are present or other challenges exist, it may be extremely difficult to pass a guidewire and deploy a stent without significant distortion. Similar issues exist with the treatment of renal arteries, which can also be difficult to access because of the difficult angles in which they may come off the descending aorta. Distortion of a stent can be particularly troublesome during the deployment of self expanding stents. For example, in cases such as the superficial femoral artery or renal arteries, the vasculature is especially tortuous, and because stents tend to have larger diameters with lower stiffness, they are prone to deformation. Consequently, there is a need for a catheter that can track to the deployment location, and once in place, have enough stiffness to ensure that the stent can be deployed with minimal distortion of the stent or the stent delivery system from the vessel anatomy.

It would be advantageous to have a delivery system catheter that has the properties of flexibility and rigidity and at times when such properties are necessary. More particularly, it would be advantageous to have a delivery system catheter that can track to a stent deployment location, and once in place, have enough stiffness to ensure that the stent can be deployed with minimal distortion of the stent from the vessel anatomy.

### BRIEF SUMMARY OF THE INVENTION

To overcome the disadvantages with existing delivery system catheters, disclosed is a delivery system catheter having selective variable stiffness. In one configuration, the delivery system catheter includes a catheter body having a proximal end, a distal end, and an interior wall surface defining a first lumen extending from the proximal end toward the distal end. A second lumen is disposed between the interior wall surface and an outer surface of the catheter body. An inflation lumen extends from the proximal end toward the distal end, the inflation lumen being disposed between the second lumen and the outer surface of the catheter body, the inflation lumen being provided to inflate a balloon that may be disposed on the distal end of the catheter body. A plurality of stiffening members is disposed around the second lumen, the plurality of stiffening members being engageable to selectively stiffen the catheter body.

According to another configuration, the second lumen is defined by a first elastomeric member and a second elastomeric member spaced apart from the first elastomeric member. The movement of the elastomeric members aid with the engagement of the stiffening members to increase the catheter's stiffness. The stiffening members can be disposed radially and/or axially to increase the stiffness.

In another configuration, the delivery system catheter includes a catheter body having a proximal end, a distal end, and interior wall surface defining a first lumen extending from the proximal end toward the distal end. A second lumen is disposed between the interior wall surface and an outer surface of the catheter body, with a plurality of stiffening members being disposed around the second lumen. Those stiffening members being engageable to selectively stiffen the catheter body. The guide catheter can include an inflation lumen that fluidly communicates with an expandable balloon disposed at the distal end of the catheter body. Optionally, a stent can be mounted to the expandable balloon.

In still another configuration, the guide catheter includes a catheter body having a proximal end, a distal end, and an interior wall surface defining a first lumen extending from the proximal end toward the distal end. A second lumen is disposed between the interior wall surface and an outer surface of the catheter body, with a plurality of stiffening members being disposed around the second lumen. A self-expanding stent can be mounted to the catheter body, while an outer sleeve at least partially surrounds that stent. An outer sleeve can be disposed upon at least a portion of the catheter body and the stent.

Methods of deploying a stent in a vessel also are disclosed. The methods comprise positioning delivery systems or delivery system catheters of the present invention within a body lumen at a treatment site. In one such method, for example, the delivery system may comprise a catheter body having a first lumen and a second lumen; a plurality of stiffening members disposed around the second lumen, the plurality of stiffening members being engageable to selectively stiffen the catheter body; and an expandable balloon in fluid communication with the first lumen. After the delivery system is position, the method comprises injecting a fluid into the second lumen to move said plurality of stiffening members into engagement to selectively stiffen the catheter body, and inflating the expandable balloon by injecting another fluid into the first lumen to deploy the stent into the vessel.

Another method of deploying a stent in a vessel comprises positioning a delivery system within a body lumen at a treatment site, wherein the delivery system, e.g., comprises a catheter body having a proximal end, a distal end, and a first lumen; a plurality of stiffening members disposed around the first lumen, the plurality of stiffening members being engageable to selectively stiffen the catheter body; a stent releasably cooperating with the distal end of the catheter body; and an outer sleeve disposed upon at least a portion of the catheter body and the stent. Such method further comprises advancing the catheter into the vessel until the distal end of the catheter body reaches the treatment site; injecting a fluid into the second lumen to move said plurality of stiffening members into engagement to selectively stiffen the catheter body; and retracting at least a portion of the outer sleeve to deploy the stent.

These and other features of the present invention will become more fully apparent from the following description and appended claims, or may be learned by the practice of the invention as set forth hereinafter.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only typical embodiments of the invention and are therefore not to be considered limiting of its scope. The invention will be described and explained with additional specificity and detail through the use of the accompanying drawings.

FIG. 1A illustrates a side view of a support catheter according to the present invention.

FIG. 1B illustrates a schematic cross-sectional view of the improved support catheter of FIG. 1A.

FIG. 2A illustrates a schematic view of a balloon expandable stent delivery system according to the present invention.

FIG. 2B illustrates a schematic cross-sectional view of the catheter of the balloon expandable stent delivery system of FIG. 2A.

FIG. 3A illustrates a schematic view of the catheter of FIGS. 2A-2B in cooperation with a vessel, wherein the catheter is in a flexible state.

FIG. 3B illustrates a schematic view of the catheter of FIGS. 2A-2B in cooperation with a vessel, wherein the catheter is in a stiffened state.

FIG. 3C illustrates a schematic view of the catheter of FIGS. 2A-2B having an expandable balloon expanded within the vessel.

FIG. 4A illustrates a side view of another delivery system catheter according to the present invention.

FIG. 4B illustrates a schematic cross-sectional view of the delivery system catheter of FIG. 4A according to the present invention.

FIG. 4C illustrates a schematic view of the delivery system catheter of FIG. 4A in cooperation with a vessel, wherein the delivery system catheter is in a flexible state.

FIG. 4D illustrates a schematic view of the delivery system catheter of FIG. 4A in cooperation with a vessel and an inflation device, wherein the delivery system catheter is in a stiffened state.

FIG. 4E illustrates a schematic view of the delivery system catheter of FIG. 4A in cooperation with a vessel, wherein the stent is shown being deployed.

FIG. 5 illustrates a schematic, partial cross-sectional side view of an alternate embodiment of a support catheter according to the present invention.

### DETAILED DESCRIPTION

Reference will now be made to figures wherein like structures will be provided with like reference designations. It is understood that the drawings are diagrammatic and schematic representations of exemplary embodiments of the invention, and are not limiting of the present invention nor are they necessarily drawn to scale.

As described herein, the delivery system catheter is employed in placing a stent within a body lumen of a patient, such as, but not limited to, the lumen of a coronary artery. However, this description is exemplary only, and it should be appreciated that embodiments of the present catheter can be employed for multiple other purposes, including, but not limited to, the deployment of balloon expandable or self-expandable stent structures in various anatomical locations, including the urinary tract, bile duct, esophagus and tracheo-bronchial tree, neurovascular, peripheral vascular, cardiac, and renal vessels, among others.

Generally, the present invention is related to improved delivery system catheters. The delivery system catheter can be configured so that a medical professional can position the delivery system catheter through tortuous pathways and track to a stent deployment location, and once in place, it will have enough stiffness to ensure that the stent can be deployed with minimal distortion of the stent from the vessel anatomy, i.e., axial dislocation of the stent or misplacement of the stent during deployment.

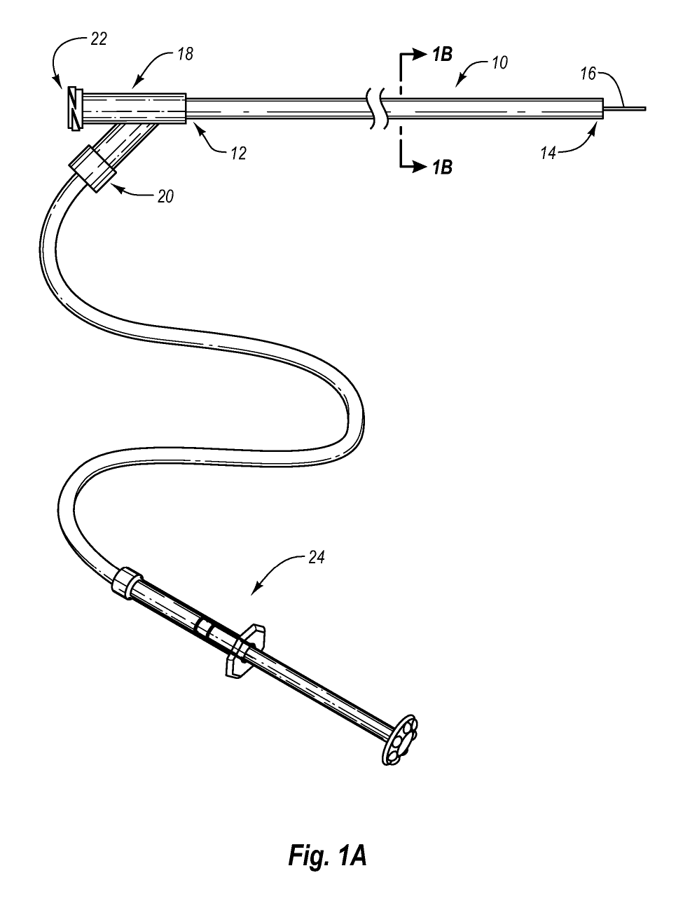

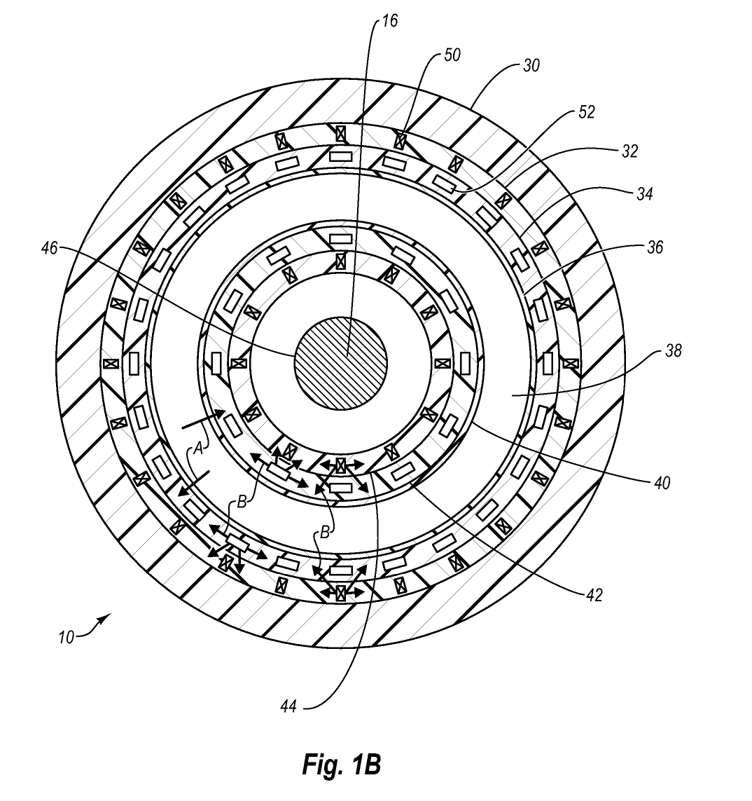

FIG. 1A illustrates a view of a catheter system that includes a support catheter 10 in fluid communication with an inflation device 24. The support catheter 10 is a stiffening support catheter that can be used, from among other applications, to track to a stent deployment location within a vessel and deploy a stent with minimal distortion of the stent from the vessel anatomy.

The support catheter 10 has additional uses. As described herein, the support catheter 10 is employed in placing a guidewire within a body lumen of a patient, such as, but not limited to, the lumen of a coronary artery. However, this description is exemplary only, and it should be appreciated that embodiments of the present catheter can be employed for multiple other purposes, including, but not limited to, the piercing of a blockage in a variety of body lumens, including the urinary tract, bile duct, esophagus and tracheo-bronchial tree, neurovascular, peripheral vascular, cardiac, and renal catheters, among others. Extra support is particularly beneficial during treatment of chronic total occlusions (“CTOs”), i.e., in recanalizing a CTO, crossing the proximal cap of CTO with a guidewire or other device or exploiting the micro channels of a CTO because the occlusion provides significant backload that is transmitted from the guidewire to the guide catheter that can lead to back out. The stiffening nature of the support catheter 10 also can be useful in reducing misplacement of a stent caused by the retraction of a delivery system sheath in the case of a self expandable stent. The support catheter 10 also can be used to aid with puncturing a heart septum with a septal puncture needle, or to support a needle used to puncture the hepatic vein and hepatic portal vein and/or the liver as part of a Transjugular Intrahepatic Portosystemic Shunt, or “TIPS” procedure. In all of these cases and others, the support catheter 10 prevents backing out of the puncture needle or guidewire, promotes accurate puncture positioning and reduces the risk of perforation or puncture of adjacent bodily structures. One skilled in the art will readily appreciate the applicability of the present invention to any aspect of the aforementioned procedures as well as other procedures in which a stiffening support catheter 10 may be useful.

As shown, the support catheter 10 has a proximal end 12 and a distal end 14; the body of the catheter or catheter body extends from the proximal end 12 toward the distal end 14. In FIG. 1A, the support catheter 10 is shown in place over a guidewire 16. The guidewire 16 extends from the proximal end 12 toward the distal end 14 and passes through a port assembly 18 disposed at the proximal end 12. The port assembly 18 can optionally be operated or function as a handle for a physician or clinician to manipulate the support catheter 10. In the illustrated configuration, the port assembly 18 includes a first port 20 usable to actuate or de-activate the support catheter 10 to decrease and/or increase the flexibility of the support catheter. 10. For instance, the inflation device 24 can be coupled to the first port 20 to fluidly communicate with the support catheter 10. The inflation device 24 can be any of a variety of different inflation structures or systems, including but not limited to, fixed or movable powered or manually operated fluid injection systems or devices typically associated with the injection of a fluid into a medical device. As shown, the inflation device 24 has the form of a manually operated syringe, however, mechanically operated or other powered systems or devices are possible. The inflation device 24 can allow pressure or volume to control the introduction of fluid into the device 24. Exemplary inflation devices are produced by B. Braun, Merit Medical, and Cook, and typically have pressure ranges of 0 to 30 atm. It is contemplated that inflation pressures of approximately 10 atm are suitable for this invention, but this may be varied to reach different stiffness levels. Systems utilizing a canister of compressed gas, e.g., compressed CO₂, as long as it satisfies the required pressure ranges, also are possible. In general, a higher pressure will yield a stiffer catheter.

With continued reference to FIG. 1A, the port assembly 18 also includes a second port 22 through which the guidewire 16 or other medical device can be placed and positioned. It will be understood that the port assembly 18 can include additional ports as needed to accomplish the desired operation and usability of the support catheter 10.

Turning now to FIG. 1B, illustrated is a cross sectional view of the support catheter 10. As shown in cross-sectional view, the support catheter 10 includes an outer jacket layer 30 that defines an outer surface of the support catheter 10 and its catheter body. Disposed within the jacket layer 30, and forming the wall of the support catheter 10, are a first stiffening coil layer 32, a first stiffening band layer 34, an annular lumen 38 (or channel), a second stiffening band layer 42, and a second stiffening coil layer 44. The first stiffening coil layer 32 and the first stiffening band layer 34 can be considered, collectively, a first stiffening layer, while the second stiffening band layer 42 and the second stiffening coil layer 44 can be considered, collectively, as a second stiffening layer. It will be understood that each of the first and second stiffening layers can include only one of the coil or band layers, however, the illustrated embodiment includes both coil and band layers.

Disposed within, and optionally defined by the interior wall surface of the second stiffening coil layer 44, is a guidewire lumen 46. This lumen 46 can receive the guidewire 16 during use of the support catheter 10.

Surrounding annular lumen 38, and optionally defining the annular lumen 38, is an outer wall 36 and an inner wall 40. The outer wall 36 and inner wall 40 are capable of flexing to enable the dimensions of the annular lumen 38 to change and thereby change the orientation and position of the layers 32, 34, 42, and 44 to change the stiffness of the support catheter 10. For instance, the outer wall 36 and inner wall 40 can be both made of an elastomeric material such as polyurethane, silicone or C-Flex®, the last of which can be manufactured by STI Components, Inc. of Morrisville, N.C., for example. More generally, the outer wall 36 and/or inner wall 40 can be fabricated from a polymeric material, a synthetic material, a natural material, or other material that provides the desired flexibility.

With continued reference to FIG. 1B, the outer jacket layer 30 defines the outer surface of the support catheter 10. As such, it may be desirable for the outer jacket layer 30, whether alone or in combination with another layer, to present a biocompatible surface to a body lumen within which it may be disposed. The outer jacket layer 30, therefore, can be made of a biocompatible material, such as a biocompatible polymeric material. Other suitable materials include polyimide, PEEK, polytetrafluorethylene, polyvinylidene fluoride, and polyamide. In addition, a biocompatible coating may be applied to any material that the catheter 10 may be composed of For example, Heparin, which prevents blood clotting, may be used as such a biocompatible coating.

The first stiffening coil layer 32 and the second stiffening coil layer 42 of the support catheter 10 can be similar and can include one or more coiled wires, such as metallic or polymeric coils, positioned in radial directions. In contrast, the first stiffening band layer 34 and the second stiffening band layer 44 can be similar and can include one or more bands, such as metallic or polymeric bands, positioned in axial directions. Although reference is made to first and second coil and band layers, it will be understood that the first and second stiffening layers generally include one or more structures that cooperate together to change the stiffness of the support catheter 10, whether or not the sub-layers, i.e., first stiffening coil layer 32, first stiffening band layer 34, second stiffening coil layer 42, and second stiffening band layer 44, include coils, bands, ribbons, or other structures. Further, and more generally, the structures forming the stiffening coil layers and the stiffening band layers, whether individually, or collectively, can be considered as stiffening members. Further, and more generally, the structures forming the stiffening coil layers and the stiffening band layers, whether individually, or collectively, can be considered as stiffening members.

Although larger ranges may work, stiffening layers could be formed from coiled wire with diameter 0.001 to 0.005 inch, and bands with thickness of 0.001 to 0.005 inch and width of 0.003 to 0.020 inch. Materials for the stiffening layers could include stainless steel, Nitinol, cobalt chrome, gold, platinum, eligloy, other metals, nylon, other polymers, and any combination thereof Spacing between bands and coil turns can vary, but would likely be in the range of 0.003 to 2.0 inches, depending on the catheter location.

Further, within the spirit of the present invention, other embodiments of the supporting catheter 10 are contemplated. For example, instead of the axially aligned or radially-aligned coils or bands of the stiffening layers 32, 34, 42, and 44, such layers may be spirally-aligned or the layers may be any combination of any of the above. Also, the number or placement of stiffening layers may vary.

The support catheter 10 of the present invention overcomes the difficulties of existing support catheters through providing structures that enable a physician or clinician to vary the stiffness of the support catheter 10 as desired. Varying the quantity of fluid within the annular lumen 38 varies the stiffness of the support catheter 10. For instance, during a procedure, the guidewire 16 can be placed in position by itself. Then, at the location and time when the guidewire 16 needs stiffening, the remaining parts of the support catheter 10 are placed in position over the guidewire 16 by advancing it over the guidewire 16 until it reaches the desired location. Then, the annular lumen 38 is filled with a fluid by way of the fluid inflation device 24 (FIG. 1A) to inflate a portion of the catheter 10. With the aid of inflation device 24, an amount of fluid is infused into the lumen 38. In general, the inflation procedure is similar to the inflation step in a procedure that would be used by a physician performing a PTCA balloon angioplasty procedure.

When the annular lumen 38 inflates, the elastomeric walls 36 and 40 press against stiffening layers 32, 34, 42, and 44. Upon inflation, the elastomeric walls 36 and 40 move in the direction of arrows A, as shown in FIG. 1B. As each material and layer of the stiffening support delivery system catheter 100 presses against the stiffening layers 32, 34, 42, and 44, they move against each other and create friction or engage with each other so that they are prohibited from recovering to their original position. Specifically, as shown in FIG. 1B, the coiled wires 50 and the bands of wire 52 of the stiffening layers 32, 34, 42, and 44 will shift in various directions, as represented by arrows B. By doing so, the coiled wires 50 and the bands of wires 52 will intermesh with each other at adjacent layers as well as within each layer. More specifically, inflation imparts forces on layers 34 and 42 such that bands of wires 52 in layers 34 and 42 move in various directions B and particularly into adjacent layers 32 and 44. In response to these forces, the coiled wires 50 move in various directions B into and particularly into adjacent layers 32 and 44. This intermeshing process is aided by the elastomeric material of walls 36 and 40 that forces its way into the gaps of any stiffening layers 32, 34, 42, and 44. Thus, the stiffening layers 32, 34, 42, and 44 become locked in their new positions, optionally even after the annular lumen 38 is deflated. Therefore, after deflation of the annular lumen 38, there is sufficient flexibility of the catheter 10 for the physician or clinician to use the guidewire 16 to advance any treatment devices over guidewire 16. In other configurations of the stiffening support delivery system catheter 10, however, the stiffening layers 32, 34, 42, and 44 may optionally return to their original positions after the annular lumen 38 is deflated. In this way, the support catheter 10 may be removed from a treatment site more easily and with less chance of bodily injury than if the annular lumen 62 had not been deflated prior to removal. To facilitate maintaining pressure in the catheter 10, a valve may be disposed on the port assembly 20 such that the inflation device 24 may be decoupled from the support catheter 10 and removed.

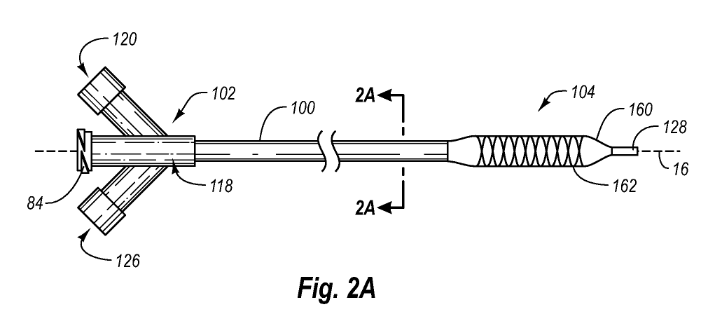

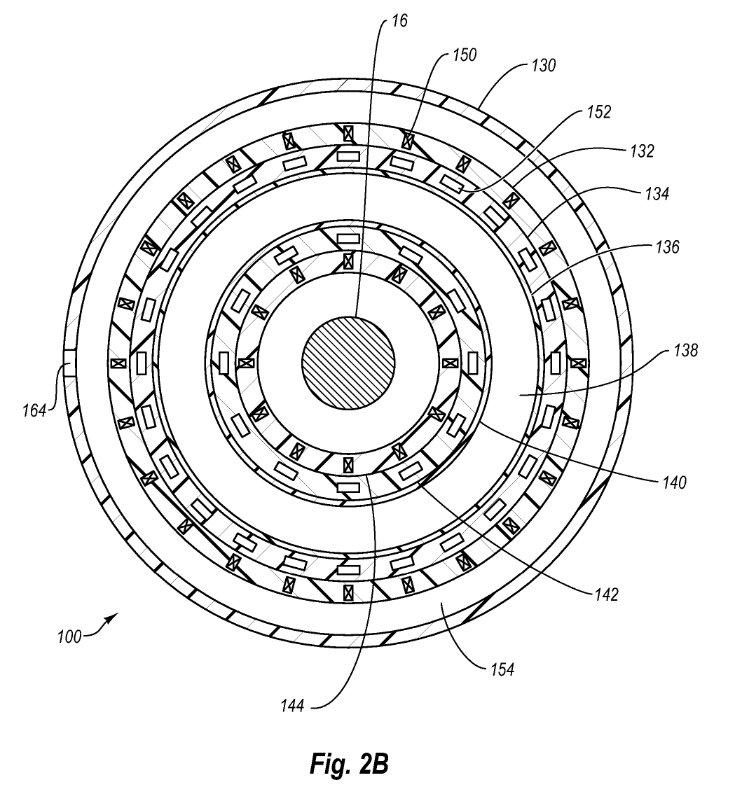

Turning now to FIGS. 2A and 2B, illustrated are schematic views of another delivery system catheter, identified by reference numeral 100. The functions, structures, and features of the delivery system catheter 10 also apply to delivery system catheter 100, and vice versa.

As depicted, delivery system catheter 100 is a balloon expandable stent delivery catheter that can be transformed from a flexible state to a stiffer state over the length of catheter 100. The catheter 100 includes a proximal end 102 and a distal end 104; the body of the catheter or catheter body extends from the proximal end 102 toward the distal end 104. The distal end 104 of the delivery system catheter 100 being transformable, allowing it to track through tortuous vessels and then allowing it to stiffen, thereby providing support against a vessel section to prevent movement during stent deployment. Formed at the distal end 104 are a flexible tip 128, such as a flexible coil, J-hook, or the like, to aid with positioning of the balloon-type delivery system catheter 100, an expandable balloon 160, and an optional stent 162 mounted or disposed around at least a portion of the expandable balloon 160.

The balloon 160 can be a polymeric balloon having sufficient flexibility to enable inflation using pressurized fluid, while being sufficiently rigid to prevent excessive inflation that could result in vessel rupture. Various types of balloon are known to those skilled in the art. For instance, the balloon can be fabricated from a polymer, a plastic, or other materials providing the desired characteristics. Further, the balloon can be fabricated from either a biocompatible material or a non-biocompatible material coated with a biocompatible coating. Suitable polymer materials include nylon, Pebax, polyurethane, PET, bioabsorbable polymers such as PLLA.

With respect to the stent 162, various types and configuration of stent can be used with the delivery system catheter 100. For instance, metallic or polymeric, coated or uncoated, expandable or self-expanding stents can be used. Some suitable stent materials include stainless steel, cobalt chrome, iron, magnesium, platinum, gold, eligiloy, Nitinol, PLLA, PEA, or combinations thereof As is known in the art, there are many other materials that are suitable for fabricating a stent.

At its proximal end 12, the delivery system catheter 100 includes a port assembly 118. In contrast to the port assembly 18, port assembly 118 includes a first port 120 useable to inflate or deflate the annular lumen 138, a second port 84 through which a physician or clinician can place the guidewire 16 or other medical devices, and a third port 126 that fluidly communicates with an expandable balloon 160 mounted or disposed at the distal end 104 of the delivery system catheter 100.

FIG. 2B shows a schematic cross-sectional view of delivery system catheter 100. The cross section of delivery system catheter 100 differs from the cross section of delivery system catheter 10 in that it further includes a balloon inflation lumen 154 between an outer jacket layer 130 and the first stiffening coil layer 132. The balloon inflation lumen 154 communicates with one or more inflation ports 164, shown in dotted lines, that fluidly communicate with the expandable balloon 160 (FIG. 2A).

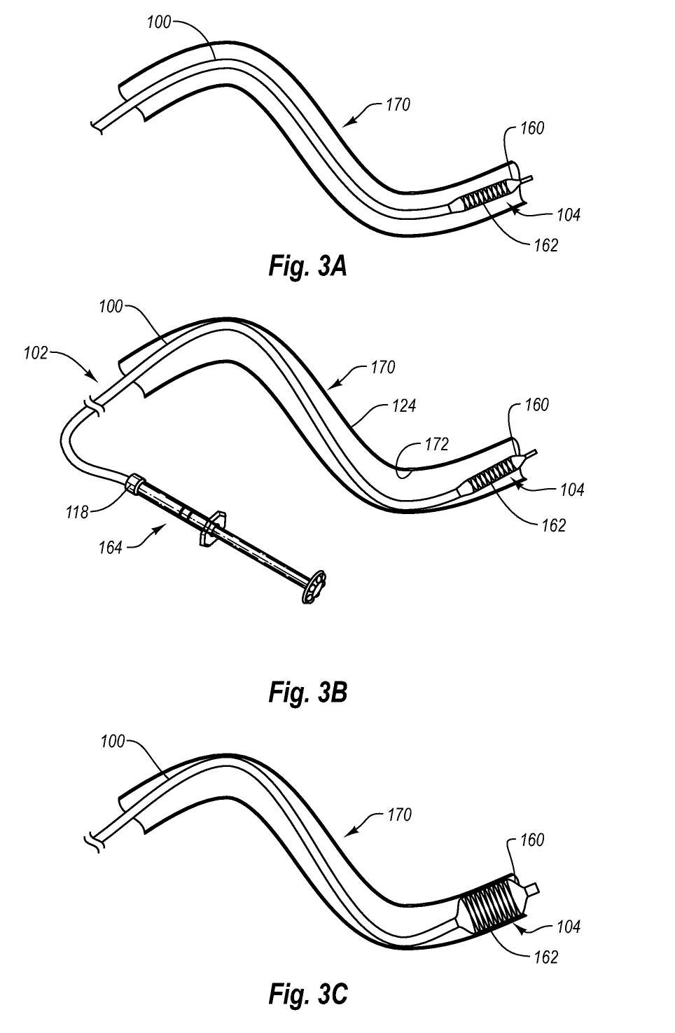

The delivery system catheter 100 of the present invention overcomes the difficulties of existing delivery system catheters through providing structures that enable a physician or clinician to vary the stiffness of the delivery system catheter 10 as desired. During a procedure, the delivery system catheter 100 is first tracked through a tortuous vessel 170 until its distal end 160 has reached the stent deployment site. As shown in FIG. 3B, the catheter 100 is then actuated or stiffened as the annular lumen 138 is inflated as described above. In doing so, the catheter 100 assumes or conforms to the shape of the vessel 170 and thereby the vessel 170 provides additional support to the catheter 100. For ease of explanation, the inflation device 24 is not included in FIGS. 3A-3C.

Once in place, and with reference to FIG. 3C, a balloon inflation device 164 mounted to port assembly 118 can be used to inflate the balloon 160. An expansion fluid can be delivered along balloon inflation lumen 154 from the port assembly catheter 118 using a syringe, pump, or other device typically used to deliver fluid to an expandable or inflatable balloon 160. More specifically, the balloon inflation device 164 delivers a quantity of fluid, whether gas, liquid, combination thereof, to the balloon 160 through the inflation ports 164 (FIG. 2B). Upon inflation of the balloon 160, the stent 162 is deployed within the vessel 170 as it contacts an interior surface 172 of walls 174. As described above, because the conformance of the stiffened catheter and the vascular anatomy results in a resistance of the stent delivery system to motion, the stent can be deployed with minimal shifting in the axial direction, which also results in accurate placement of the stent 162.

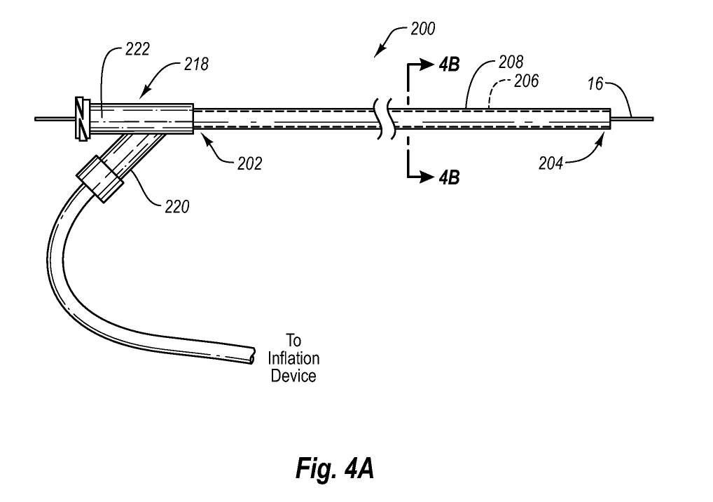

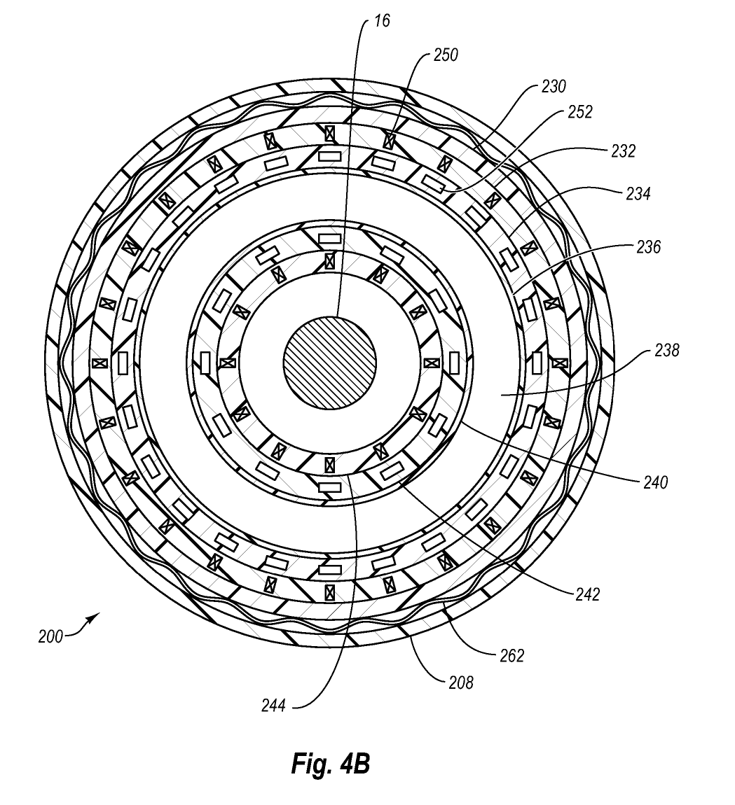

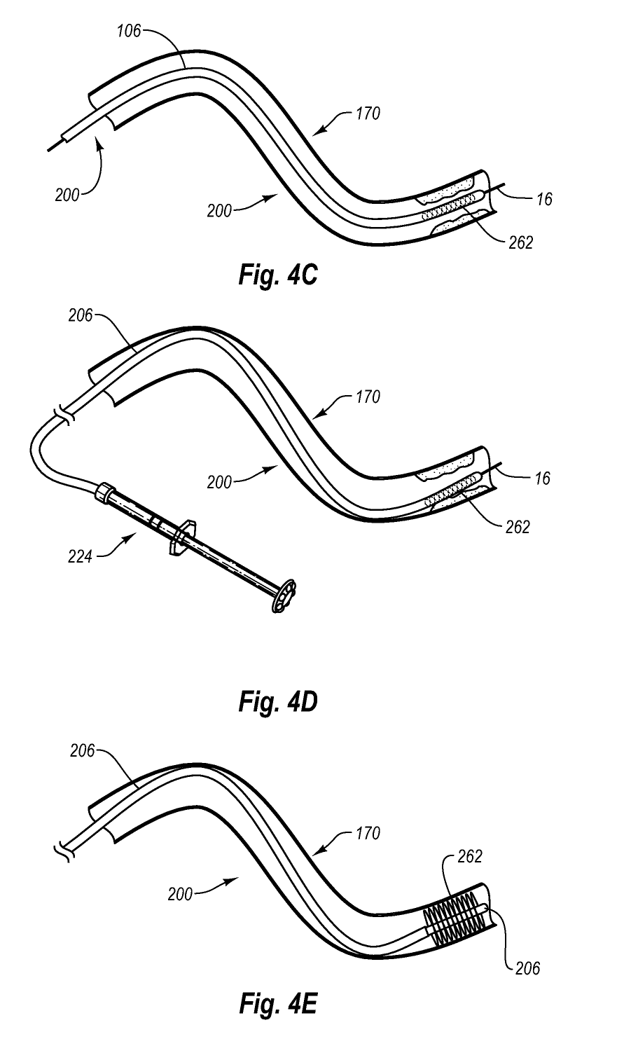

Turning now to FIGS. 4A-4D, illustrated schematically is another configuration of the delivery system catheter of the present invention. The delivery system catheter 200 is configured to cooperate with a self-expanding stent 262 rather than an expandable stent, as described with respect to FIGS. 2A-3C. Although different stents are used, the disclosure, functions, structures, and features of catheters 10 and 100 are also applicable to the delivery system catheter 200.

As depicted in FIG. 4A, the delivery system catheter 200 can be used to deliver a self-expanding stent 262. The delivery system catheter 200 is in fluid communication with an inflation device 224 (FIG. 4D), which can be similar to the other inflation devices described herein. As shown, the delivery system catheter 200 has a proximal end 202, a distal end 204, and a first catheter assembly 206 extending therebetween; the body of the first catheter assembly or catheter body extends from the proximal end 202 toward the distal end 204. Disposed upon and surrounding at least a portion of the first catheter assembly 206, and extending from the distal end 204 toward the proximal end 202, is an outer sleeve 208. The self-expanding stent 262 is disposed between the first catheter assembly 206 and the outer sleeve 208, with such positioning retaining the self-expanding stent 262 until deployment.

Generally, the first catheter assembly 206 can have the same structures, functions, and features to the catheter 10 described herein and so the description of catheter 10 also applies to the first catheter assembly 206 of the delivery system catheter 200. As such, and with continued reference to FIG. 4A, the delivery system catheter 200 is shown in place over a guidewire 16. The guidewire 16 extends from the proximal end 202 toward the distal end 204 and passes through a port assembly 218 disposed at the proximal end 202 and mounted to the first catheter assembly 206. The port assembly 218 can optionally be operated or function as a handle for a physician or clinician to manipulate the delivery system catheter 200. In the illustrated configuration, the port assembly 218 includes a first port 220 usable to actuate or de-activate the first catheter assembly 206 to decrease and/or increase the flexibility of the first catheter assembly 206 and so the delivery system catheter 200. For instance, the inflation device 224 can be coupled to the first port 220 to fluidly communicate with the first catheter assembly 206 of the delivery system catheter 200.

With continued reference to FIG. 4A, the port assembly 218 also includes a second port 222 through which the guidewire 16 or other medical device can be placed and positioned. It will be understood that the port assembly 218 can include additional ports as needed to accomplish the desired operation and usability of the delivery system catheter 200.

Turning now to FIG. 4B, illustrated is a cross-sectional view of the delivery system catheter 200. As shown, the first catheter assembly 206 includes a jacket layer 30 that defines an outer surface of the delivery system catheter 10 and its catheter body. Disposed within the jacket layer 230, and forming the wall of the delivery system catheter 200, are a first stiffening coil layer 232, a first stiffening band layer 234, an annular lumen 238 (or channel), a second stiffening band layer 242, and a second stiffening coil layer 244. The first stiffening coil layer 232 and the first stiffening band layer 234 can be considered, collectively, a first stiffening layer, while the second stiffening band layer 242 and the second stiffening coil layer 244 can be considered, collectively, as a second stiffening layer. It will be understood that each of the first and second stiffening layers can include only one of the coil or band layers, however, the illustrated embodiment includes both coil and band layers.

Disposed within, and optionally defined by the interior surface of the second stiffening coil layer 244, is a guidewire lumen 246. This lumen 246 can receive the guidewire 16 during use of the delivery system catheter 200.

Surrounding annular lumen 238, and optionally defining the annular lumen 238, is an outer wall 236 and an inner wall 240. The outer wall 236 and inner wall 240 are capable of flexing to enable the dimensions of the annular lumen 238 to change and thereby change the orientation and position of the layers 232, 234, 242, and 244 to change the stiffness of the delivery system catheter 200. For instance, the outer wall 236 and inner wall 240 can be fabricated from a polymeric material, a synthetic material, a natural material, or other material that provides the desired flexibility.

Disposed upon and/or surrounding at least a portion of the first catheter assembly 206 are the stent 262 and the outer sleeve 208. The stent 262 can be disposed upon a portion of the jacket layer 230, while the sleeve 208 can cover the stent 262 and a portion of the jacket layer 230. The stent 262 can be fabricated from a variety of different materials, such as shape memory materials, including, but not limited to, shape memory alloys, such as Nitinol, copper-zinc-aluminum, and copper-aluminum-nickel alloys. Some of these shape memory alloys may not be biocompatible, and may therefore require protective encapsulation to be used in a medical application. It also is possible to form the stent from shape memory polymers, such as Veriflex or other suitable SMPs. Optionally, the stent 262 can be coated with a variety of different coatings known to those skilled in the art, including, but not limited to, therapeutic agents, such as anti-proliferative, anti-inflammmatory, antineoplastic, antiplatelet, anti-coagulant, anti-fibrin, antithrombonic, antimitotic, antibiotic, antiallergic and/or antioxidant compounds.

The sleeve 208 covering the stent 262 is moveably coupled to the first catheter assembly 206 and can be moved distally or proximally relative to the stent 262. In particular, the sleeve 208 can be moved proximally, i.e., toward the proximal end 202, during a procedure to release the stent 262. Depending upon the particular flexibility or rigidity properties, the sleeve 208 can tightly surround the stent 262 and form-fit to the stent 262 and/or the first catheter assembly 206.

The sleeve 208 can be fabricated from a variety of different materials and can optionally be coated with a variety of different agents or drugs. For instance, the sleeve 208 can be fabricated from a polymeric material and coated with a biocompatible coating. In another configuration, the sleeve 208 is fabricated from a biocompatible material. In still another configuration, the sleeve 208 can be fabricated from a metallic material. Suitable sleeve materials may include polyimide, nylon, polyamide, polyethylene, PTFE, FEP, PEEK, PVDF and other materials that are known in the art.

Turning now to FIGS. 4C-4E, the operation and use of the catheter 200 is illustrated. As shown schematically in FIG. 4C, the catheter 200 can be steered through the tortuous anatomy of a patient through the vessel 170 until it reaches the lesion 172 or other obstruction as it follows the guidewire 16. After the catheter 200 reaches the treatment site 172, the annular lumen 238 (FIG. 4B) is then inflated as described above. As shown in FIG. 4D, an inflation device 224 is shown attached to the proximal end 202 for inflating the annular lumen 238. In doing so, the first catheter assembly 206, and hence the catheter 200, assumes or forms to the shape of the vessel 170, and thereby, the vessel 170 itself provides additional support to the catheter 200.

FIG. 4E shows a schematic view of the last step in deployment of the stent 262. That is, after the annular lumen 328 (FIG. 4B) is inflated to stiffen the first catheter assembly 206, the outer sleeve 208 is moved proximally to deploy the stent 262. The outer sleeve 208 can be retracted a sufficient distance to deploy the stent 262 or can be completely removed, depending upon the particular configuration of proximal end 202. During movement of the outer sleeve 208, the first catheter assembly 206 will maintain its shape without the additional support of the outer sleeve 208. Thus, the catheter 200 allows the stent 262 to deploy in a uniform manner within the substantially free shape of the vessel 170. Accordingly, with the supportive delivery system catheter 200 providing support and preventing movement due to its conformance with the vessel 170 walls which provide added support, the stent 262 can be deployed with minimal movement of the stent 262, which results in accurate placement of the stent 262.

Following deployment of stent 262, as with the other embodiments of the catheter, the fluid is removed from annular lumen 238 sufficiently to decrease catheter stiffness. In this way, the catheter 200 may be removed from a treatment site more easily and with less chance of bodily injury than if the annular lumen 238 had not been deflated prior to removal.

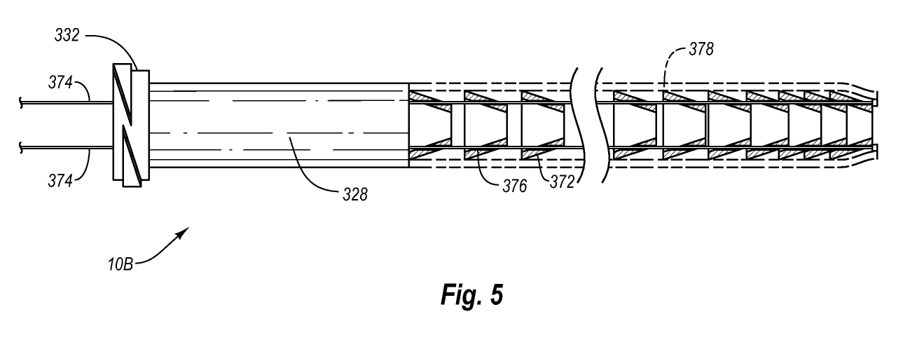

Note that there are other stiffening support catheters that will work with the delivery system catheters and methods of the present invention disclosed herein. For example, FIG. 5 shows a schematic, partial cross-sectional side view of an alternate embodiment of a support catheter 10B according to the present invention. Support catheter 10B comprises a plurality of conically-shaped members or truncated cones 372 that are movably linked by two cables 374, which pass through holes or respective passageways 376 in each conically-shaped member 372. The plurality of conically-shaped members 372 are situated on the port assembly 328, which contains a first port 332, out of which emanate the two cables 374.

The truncated cones 372 are generally concentric in that they fit inside one another, as shown in FIG. 5. When the two cables 374 are pulled from the first port 332, the truncated cones 372 are forced closer together, and in turn, the arrangement and thereby the support catheter 10B becomes stiffer. Accordingly, in practice, the alignment of truncated cones 372 is used as a support catheter 10B just as support catheter 10 or the other catheters of the delivery system catheters can be used as described above. Note that the cables 374 can be any thin elongated material suitable for working inside the body, such as wire, line, cord, or the like.

Once a guidewire 16 is in position, the catheter 10B can be advanced over the guidewire 16. The relative stiffness of the support catheter 10B can be adjusted by the medical professional by means of the cables 374. Note that although two cables 374 are shown in the embodiment of FIG. 5, only one cable 374 is necessary and more than two cables 374 may be utilized. Also note that the truncated cones 72 of support catheter 10B of FIG. 5 are shown in various modes of alignment. That is, those truncated cones 372 on the distal end of catheter 10B are shown in relatively tight engagement, whereas those shown on the proximal end of the catheter 10B are shown separated from each other for illustrative purposes. Accordingly, in practice, as understood by one skilled in the art, the truncated cones 372 would be aligned similar to those shown on the distal end of catheter 10B, as friction between the truncated cones 372 and the cables 374 holds the catheter 10B together and allows it to both advance over a guidewire 16, but also to provide support to the guidewire 16. The truncated cones 372 also may have modified surfaces to increase the friction therebetween. That is, they may be coated with a material having a higher friction than the core, such as silicone, or they may be roughened or knurled to increase friction.

The truncated cones 372 of catheter 10B may be manufactured from any number of materials. For example, such suitable materials may include metals, metal alloys, polymers, synthetic materials, natural materials, combinations thereof, or other materials that provide the desired stiffness and/or biocompatibility. Suitable metals include stainless steel, Nitinol, elgiloy, and cobalt chromium. Suitable polymers include PTFE, ePTFE, PEEK, polyimide, and Pebax. Further, catheter 10B may further comprise an outer jacket layer 378, as shown in phantom lines in FIG. 5. The outer jacket layer 378 may be made of a biocompatible material, such as a biocompatible polymeric material. Suitable biocompatible polymeric materials for the outer jacket layer 378 may include polyimide, PEEK, polytetrafluorethylene, polyvinylidene fluoride, and polyamide, although other materials are possible.

As part of a delivery system catheter of the present invention, a self-expanding stent may be positioned on the distal end of catheter 10B between the layer of truncated cones 372 and an outer jacket layer 378. Alternatively, a balloon and stent may be positioned on the distal end of the catheter 10B, with a balloon inflation lumen provided to inflate the balloon.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description.

What is claimed is:

1. A method of deploying a stent in a vessel, the method comprising:
positioning a delivery system within a body lumen at a treatment site, the delivery system comprising:
a catheter body having a first lumen and a second lumen;
a plurality of stiffening members disposed around the second lumen, the plurality of stiffening members being engageable to selectively stiffen the catheter body; and
an expandable balloon in fluid communication with the first lumen;
injecting a fluid into the second lumen to move said plurality of stiffening members into engagement to selectively stiffen the catheter body; and
inflating the expandable balloon by injecting another fluid into the first lumen to deploy the stent into the vessel.

2. A method of deploying a stent in a vessel, the method comprising:
positioning a delivery system within a body lumen at a treatment site, the delivery system comprising:
a catheter body having a first lumen and a second lumen;
a plurality of stiffening members disposed around the second lumen, the plurality of stiffening members being engageable to selectively stiffen the catheter body; and
an expandable balloon in fluid communication with the first lumen; and
inflating the expandable balloon by injecting a fluid into the first lumen to deploy the stent into the vessel.

3. A method of deploying a stent in a vessel of claim 2, wherein prior to inflating the expandable balloon, the method further comprises the step of:
injecting a fluid into the second lumen to move said plurality of stiffening members into engagement to selectively stiffen the catheter body.

4. A method of deploying a stent in a vessel, the method comprising:
positioning a delivery system within a body lumen at a treatment site, the delivery system comprising:
a catheter body having a proximal end, a distal end, and a first lumen;
a plurality of stiffening members disposed around the first lumen, the plurality of stiffening members being engageable to selectively stiffen the catheter body; and
a stent releasably cooperating with the distal end of the catheter body;
and
an outer sleeve disposed upon at least a portion of the catheter body and the stent;
advancing the catheter into the vessel until the distal end of the catheter body reaches the treatment site; and
retracting at least a portion of the outer sleeve to deploy the stent.

5. A method of deploying a stent in a vessel of claim 4, wherein prior to retracting at least a portion of the outer sleeve, the method further comprises the step of:
injecting a fluid into the second lumen to move said plurality of stiffening members into engagement to selectively stiffen the catheter body.

6. A method of deploying a stent in a vessel, the method comprising:
positioning a delivery system within a body lumen at a treatment site, the delivery system comprising:
a catheter body having a proximal end, a distal end, and a first lumen;
a plurality of stiffening members disposed around the first lumen, the plurality of stiffening members being engageable to selectively stiffen the catheter body; and
a stent releasably cooperating with the distal end of the catheter body;
and
an outer sleeve disposed upon at least a portion of the catheter body and the stent;
advancing the catheter into the vessel until the distal end of the catheter body reaches the treatment site;
injecting a fluid into the second lumen to move said plurality of stiffening members into engagement to selectively stiffen the catheter body; and
retracting at least a portion of the outer sleeve to deploy the stent.

7. A delivery system catheter comprising:
a catheter body having a proximal end, a distal end, and an interior wall surface defining a first lumen extending from the proximal end toward the distal end;
a second lumen disposed between the interior wall surface and an outer surface of the catheter body;
a plurality of stiffening members disposed around the second lumen, the plurality of stiffening members being engageable to selectively stiffen the catheter body; and
an expandable balloon disposed at the distal end of the catheter body.

8. The delivery system catheter as recited in claim 7, further comprising an inflation lumen extending from the proximal end toward the distal end, the inflation lumen being in fluid communication with the expandable balloon.

9. The delivery system catheter as recited in claim 7, further comprising a stent releasably coupled to the expandable balloon.

10. The delivery system catheter as recited in claim 7, further comprising a port assembly disposed at the proximal end.

11. The delivery system catheter as recited in claim 7, further comprising a first elastomeric member and a second elastomeric member spaced apart from the first elastomeric member, the first and second elastomeric members defining the second lumen.

12. The delivery system catheter as recited in claim 7, wherein at least one of the plurality of stiffening members comprises a radially orientated stiffening member and at least one of the plurality of stiffening members comprises an axially orientated stiffening member.

13. The delivery system catheter as recited in claim 12, wherein the radially orientated stiffening member comprises a stiffening band or stiffening coil and the axially orientated stiffening member comprises a stiffening band or stiffening coil.

14. A delivery system catheter comprising:
a catheter body having a proximal end, a distal end, and an interior wall surface defining a first lumen extending from the proximal end toward the distal end;
a second lumen disposed between the interior wall surface and an outer surface of the catheter body;
a plurality of stiffening members disposed around the second lumen, the plurality of stiffening members being engageable to selectively stiffen the catheter body; and
a stent releasably cooperating with the distal end of the delivery system catheter.

15. The delivery system catheter as recited in claim 14, further comprising an outer sleeve disposed upon at least a portion of the catheter body and the stent.

16. The delivery system catheter as recited in claim 15, wherein the outer sleeve is moveable relative to the catheter body and the stent.

17. The delivery system catheter as recited in claim 14, further comprising a first elastomeric member and a second elastomeric member spaced apart from the first elastomeric member, the first and second elastomeric members defining the second lumen.

18. The delivery system catheter as recited in claim 14, wherein at least one of the plurality of stiffening members comprises a radially orientated stiffening member and at least one of the plurality of stiffening members comprises an axially orientated stiffening member.

19. The delivery system catheter as recited in claim 18, wherein the radially orientated stiffening member comprises a stiffening band or stiffening coil and the axially orientated stiffening member comprises a stiffening band or stiffening coil.
